(19) United States
(12) Patent Application Publication
(10) Pub. No.: US 2013/0137114 A1
(43) Pub. Date: May 30, 2013

(54) Bioprobe, Method of Preparing the Bioprobe, and Analysis Apparatus and Method Using the Bioprobe

(71) Applicant: Industry-Academic Cooperation Foundation, Yonsei University, Seoul (KR)

(72) Inventors: Seungjoo Haam, Seoul (KR); Jin-Suck Suh, Seoul (KR); Yong-Min Huh, Seoul (KR); Jaemoon Yang, Seoul (KR); Eun-Kyung Lim, Seoul (KR); Yoonah Kang, Seoul (KR)

(73) Assignee: Industry-Academic Cooperation Foundation, Yonsei University, Seoul (KR)

(21) Appl. No.: 13/746,205

(22) Filed: Jan. 21, 2013

Related U.S. Application Data
(63) Continuation of application No. 12/681,095, filed on Mar. 31, 2010.

(30) Foreign Application Priority Data
Dec. 23, 2008 (KR) 10-2008-0132355

(51) Int. Cl. G01N 33/543

(52) U.S. Cl. CPC G01N 33/54346
USPC 435/7.1

(57) ABSTRACT

The present invention relates to a bioprobe including a substrate and inorganic nanoparticles attached to the surface of the substrate, a method of preparing the bioprobe, and an analysis apparatus and method using the bioprobe. In the bioprobe according to the present invention, inorganic nanoparticles introduced to the substrate serve as a linker to which a target-specific substance such as an antibody can be bound, and they also increase the surface area of the substrate, thus increasing a surface area where a target substance to be detected can contact the substrate. In this regard, the bioprobe can be effectively used for detection, dosing, or analysis of various biomolecules or other chemical substances.

### CROSS REFERENCE TO RELATED APPLICATION

This application is a continuation of pending U.S. patent application Ser. No. 12/681,095, filed Mar. 31, 2010, entitled “Bioprobe, Method of Preparing the Bioprobe, and Analysis Apparatus and Method Using the Bioprobe,” which application claims the benefit of International Application No. PCT/KR2009/001634, filed Mar. 31, 2009, entitled “Bioprobe, Method of Preparing the Bioprobe, and Analysis Apparatus and Method Using the Bioprobe,” which application claims the benefit of Korean Application Serial No. KR 10-2008-0132355, filed Dec. 23, 2008, which are each incorporated herein in their entirety by reference.

### TECHNICAL FIELD

The present invention generally relates to a bioprobe, a method of preparing the bioprobe, and an analysis apparatus and method using the bioprobe.

### BACKGROUND ART

Nano Technology (NT), which manipulates and controls a substance at the atomic or molecular scale, is suitable for the creation of new substances or new devices, and in this regard, can be applied in various fields such as electronics, materials, communications, mechanics, medicine, agriculture, energy, and the environment.

NT is currently being developed in a variety of ways and can be roughly classified into three fields: the first one concerns technology for synthesizing new substances and materials of micro sizes from nano materials, the second one concerns technology for manufacturing nano devices which have particular functions through binding or arrangement of nano-size materials, and the third one concerns nano-biotechnology for applying NT to the biotechnology field.

In nano-biotechnology, nanoparticles have been variously applied in detection, dosing, and separation of bio-molecules related to various diseases due to their unique characteristics at the nano scale and their ability to be easily functionalized by using various organic/inorganic compounds.

For example, Korean Patent Publication No. 2008-11856 discloses a method for analyzing a target substance by using a single-stranded nucleic acid that is specifically bound to various biomolecules and a gold nanoparticle that can form a complex with the single-stranded nucleic acid. The gold nanoparticle has a red color when well dispersed in a medium in an aqueous phase, but its color changes to violet when it becomes an aggregate of particles. In the disclosed technique, the single-stranded nucleic acid is added to the surface of a gold nanoparticle by using the foregoing principle to cause a biomolecule reaction, leading to an aggregation of gold nanoparticles, whereby analysis can be achieved by observing a change in color of a solution.

Further, Korean Patent Publication No. 2008-60841 discloses a technique in which a substrate having dispersed and bound gold nanoparticles is prepared in-situ, the gold nanoparticles are three-dimensionally distributed on the substrate, and thus densification and fixation of biomolecules, especially proteins, is possible by means of the three-dimensional distribution.

In this technique, however, since the gold nanoparticles exist in a dispersed state in a high-polymer medium, a target-specific substance such as an antibody cannot be fixed and thus the types of target substances that can be detected are limited.

### DISCLOSURE OF INVENTION

### Technical Problem

The present invention is designed to provide a bioprobe capable of precisely detecting, dosing, and separating various target substances, e.g., even a small amount of a target substance having a very small size, a method of preparing the bioprobe, and an analysis apparatus and method using the bioprobe.

### Technical Solution

To achieve the foregoing object, there is provided a bioprobe including a substrate and inorganic nanoparticles attached to a surface of the substrate.

To achieve the foregoing object, there is also provided a method of preparing a bioprobe. The method includes a first step of introducing a functional group onto a substrate by bringing the substrate into contact with a functional-group containing compound, and a second step of binding inorganic nanoparticles onto the substrate by brining the functional-group introduced substrate into contact with the inorganic nanoparticles.

To achieve the foregoing object, there is also provided an analysis apparatus including a bioprobe according to the present invention and a measurement device capable of detecting a signal emitted from the bioprobe.

To achieve the foregoing object, there is also provided an analysis method including (1) a first step of brining a bioprobe according to the present invention into contact with an analysis target specimen and (2) a second step of detecting a signal emitted from the bioprobe which has passed through (1).

### Advantageous Effects

In the bioprobe according to the present invention, inorganic nanoparticles introduced to the substrate serve as a linker to which a target-specific substance such as an antibody can be bound, and they also increase the surface area of the substrate, thus increasing a surface area where a target substance to be detected can contact the substrate. In this regard, the bioprobe can be effectively used for detection, dosing, or analysis of various biomolecules or other chemical substances.

### BRIEF DESCRIPTION OF DRAWINGS

FIG. 1 is a diagram showing a process of preparing a bioprobe to which a tissue-specific binding component is bound according to an embodiment of the present invention;

FIG. 2 is a Transmission Electron Microscope (TEM) picture of gold nano particles synthesized according to an embodiment of the present invention;

FIG. 3 shows an FT-IR spectrum of a prepared gold nanoparticles-bound substrate according to an embodiment of the present invention;

FIG. 4 shows UV-vis absorption spectrums of an aminated substrate and a gold nanoparticles-bound substrate according to an embodiment of the present invention;

FIG. 5 shows a light-scattered image of gold nanoparticles obtained by using a dark field microscope according to an embodiment of the present invention;

FIGS. 6A through 6D show AFM analysis results of a prepared bioprobe according to an embodiment of the present invention; and

FIGS. 7A through 7C and 8 show cancer cell detectivity analysis results of a prepared bioprobe according to an embodiment of the present invention.

### BEST MODE FOR CARRYING OUT THE INVENTION

The present invention relates to a bioprobe including a substrate and inorganic nanoparticles attached to the surface of the substrate.

Hereinafter, the bioprobe according to the present invention will be described in detail.

The bioprobe according to the present invention includes inorganic nanoparticles attached to a predetermined substrate. In the bioprobe according to the present invention, the inorganic nanoparticles serve as a linker to which a target-specific substance such as an antibody can be bound, and they also increase the roughness of the substrate, thereby increasing the entire surface area. Thus, if, for example, a target-specific substance is bound to the bioprobe according to the present invention for application to various bioassays, a target substance having a very small size (biomolecules, cells, or other chemical substances) can be precisely detected and separated from a specimen including a very small amount of the target substance.

The type of substrate that can be used in the present invention is not specifically limited if it is generally used in this field, and for example, a glass, a silicon substrate, quartz, metal, a high-polymer film (e.g., a cycloolefin polymer, poly(alkyl (meta)acrylate), polystyrene, polyethylene, polypropylene, polyester, polyamino acid, polyethyleneimine, polyacrylic acid, etc.), etc. can be used alone or in a mixture of two or more kinds thereof. In the present invention, more preferably, a glass substrate, a silicon substrate, or the foregoing substrate on which siliconization is performed (e.g., siliconized glass slide), but not being limited thereto, may be used as the substrate.

The bioprobe according to the present invention includes inorganic nanoparticles bound to the foregoing substrate. The inorganic nanoparticles can serve as a linker for introduction of various target-specific substances (e.g., antibodies) to the substrate and also increase the surface roughness of the substrate. Therefore, the bioprobe according to the present invention to which the inorganic nanoparticles are attached has an average roughness of preferably 10 nm 1 μm. The term ‘average roughness’ used herein means an average height from the central line of a cross-sectional profile of the bioprobe to the top of the cross-sectional profile, and the average roughness can be measured by using a Scanning Probe Microscope (SPM) such as an Atomic Force Microscope (AFM). In the present invention, if the average roughness is less than 10 nm, the surface area increase of the substrate is degraded, thus reducing the ability to detect biomolecules or other chemical substances. An average roughness exceeding 1 μm may hinder the flow of a specimen including biomolecules or a detection substance, thereby reducing the detection efficiency.

In the bioprobe according to the present invention, it is preferable that the number of inorganic nanoparticles is 10 50 per unit area (μm²) of the substrate. If the number of inorganic nanoparticles is less than 10 per unit area, the number of target-specific substances able to be bound to the inorganic nanoparticles is reduced, thus degrading the detection efficiency. If the number exceeds 50, the performance of the bioprobe may be degraded for reasons such as reduction of the surface area.

The term ‘inorganic nanoparticle’ used herein means a particle which has a nano scale and the entirety or main component of which is composed of inorganic substances. The detailed type of the inorganic nanoparticle is not specifically limited if it can be bound to the surface of the substrate in an exposed state and can provide a site to which a target-specific substance can be bound. In the present invention, for example, a metal nanoparticle or a magnetic nanoparticle may be used as the inorganic nanoparticle. The type of metal nanoparticle may be a gold (Au) nanoparticle, a platinum (Pt) nanoparticle, a silver (Ag) nanoparticle, or a copper (Cu) nanoparticle, and the type of magnetic nanoparticle may be a metal substance, a magnetic substance, or a magnetic alloy. Examples of the metal substance may be of the same type as the metal nanoparticle, examples of the magnetic substance may be one or more selected from a group consisting of Co, Mn, Fe, Ni, Gd, Mo, MM₂O₄, and MₓM_{y} (M and M independently indicate Co, Fe, Ni, Mn, Zn, Gd, or Cr, and x and y satisfy ‘0<x≦3’ and ‘0<y≦5’ respectively), and examples of the magnetic alloy may be one or more selected from a group consisting of CoCu, CoPt, FePt, CoSm, NiFe, and NiFeCo, without being limited thereto.

In the present invention, the inorganic nanoparticle has an average diameter of 1 nm 100 nm, preferably 1 nm 50 nm, and more preferably 1 nm 20 nm. If the average diameter is less than 1 nm, the binding efficiency or the surface area increase of a target-specific substance may be reduced. If the average diameter exceeds 100 nm, the surface area is reduced, degrading the performance of the bioprobe.

In the present invention, the inorganic nanoparticle may be bound to the surface of the substrate by using one or more functional groups selected from a group consisting of an amine group and a thiol group as a medium. In this way, the inorganic nanoparticle is directly bound to the substrate in an exposed state by using a predetermined functional group as a medium, thereby increasing the surface area of the substrate and providing a site for binding with a target-specific molecule. A method for introducing the functional group to the substrate is not specifically limited, and for example, if the substrate is a glass substrate, a silicon substrate, or a siliconized substrate, the functional group may be introduced by treating the substrate of the present invention with a general silane compound including the functional group.

The bioprobe according to the present invention may further include a target-specific substance bound to the inorganic nanoparticle. In this case, the inorganic nanoparticles included in the bioprobe according to the present invention may serve as a linker for connecting the target-specific substance with the substrate.

The term ‘target-specific substance’ used herein means a biomolecule or other chemical substances which are specifically bindable to a particular component to be detected, and examples thereof may include one kind or two or more kinds of an antigen, an antibody, RNA, DNA, hapten, avidin, streptavidin, neutravidin, protein A, protein G, lectin, selectin, a radioactive isotope marking substance, aptamer, and a substance that is specifically bindable to a tumor marker, without being limited thereto.

Among those examples of the target-specific substance, a substance that is specifically bindable to a tumor marker can be usefully used when the bioprobe according to the present invention is applied to the diagnosis of various diseases related to tumors such as stomach cancer, lung cancer, breast cancer, ovarian cancer, liver cancer, bronchogenic carcinoma, laryngeal cancer, pancreatic cancer, bladder cancer, colon cancer, cervical cancer, and the like. The term ‘tumor marker’ used herein means a specific substance which is not almost or entirely produced in a normal cell, whereas it is specifically revealed or secreted in a tumor cell. When the substance which is specifically bindable to the tumor marker is employed in the bioprobe according to the present invention, it can be usefully used for the diagnosis of tumors. In the art, not only various tumor markers but also substances that are specifically bindable to the tumor markers are known.

When the tumor marker is an antigen, a substance that is specifically bindable to the antigen can be introduced to the bioprobe according to the present invention, an example of which may be a receptor or an antibody that is specifically bindable to the antigen. Examples of an antigen and a receptor or an antibody that is specifically bindable to the antigen may include an Epidermal Growth Factor (EGF) and anti-EGFR (e.g., cetuximab), C2 of synaptotagmin and phosphatidylserine, annexin V and phosphatidylserine, integrin and a receptor thereof, a Vascular Endothelial Growth Factor (VEGF) and a receptor thereof, angiopoietin and a Tie2 receptor, somatostatin and a receptor thereof or vasointestinal peptide, a carcinoembryonic antigen (colon cancer marking antigen) and Herceptin (Genentech, USA), HER2/neu antigen (breast cancer marking antigen) and Herceptin, a prostate-specific membrane antigen (prostatic cancer marking antigen), Rituxan (IDCE/Genentech, USA), and a receptor thereof, without being limited thereto.

A representative example of a tumor marker which is a receptor may be a folic acid receptor revealed in an ovarian cancer cell. A substance which is specifically bindable to the receptor (folic acid in the case of a folic acid receptor) can be introduced to the bioprobe according to the present invention, and an example thereof may be an antigen or an antibody which is specifically bindable to the receptor.

As such, an antibody is a particularly preferable tissue-specific bindable substance in the present invention, and the antibody includes a polyclonal antibody, a monoclonal antibody, and an antibody fragment herein. The antibody has a feature of being able to be selectively and stably bound only to a specific target, and —NH₂ of lysine, —SH of cysteine, and —COOH of aspartic acid and glutamic acid in an Fc region of the antibody can be usefully used to bind the antibody to the inorganic nanoparticles of the bioprobe according to the present invention.

The antibody can be commercially acquired or may be prepared by a method well-known in this field.

The term ‘nucleic acid’ used herein includes an antigen, and the RNA and DNA which code the antigen, the receptor, or a part thereof. Since nucleic acids form a base pair between complementary sequences, a nucleic acid having a specific base sequence can be detected by using a nucleic acid having a base sequence which is complementary to the specific base sequence. The enzyme, the antigen, and the nucleic acid having a complementary base sequence to the nucleic acid which codes the antigen or the receptor can be used in the bioprobe according to the present invention.

The nucleic acid has a functional group such as —NH₂, —SH, or —COOH at 5 and 3′ ends thereof, and the functional group can be usefully used in the introduction of a nucleic acid to the inorganic nanoparticles of the present invention.

Such a nucleic acid can be synthesized by using a standard method well known in the art, e.g., an automatic DNA synthesizer which may be purchased from Bio-Search, Applied Biosystems, etc. For example, phosphorothioate oligonucleotide can be synthesized by using a method disclosed in the literature (Stein et al. Nucl. Acids Res. 1998, vol. 16, p. 3209). Methylphosphonate oligonucleotide can be prepared by using a regulated glass polymer support (Sarin et al. Proc. Natl. Acad. Sci. U.S.A. 1998, vol. 85, p. 7448).

A method of preparing the bioprobe according to the present invention is not specifically limited. The bioprobe may be prepared, for example, by a method including a first step of introducing a functional group to a substrate by brining the substrate into contact with a functional-group containing compound; and

a second step of binding inorganic nanoparticles onto the substrate by bringing the functional-group introduced substrate into contact with the inorganic nanoparticles.

The first step of the preparation method is a step of bringing the substrate into contact with the functional-group containing compound in order to provide a site on the substrate included in the bioprobe for absorption of the inorganic nanoparticles.

A detailed type of the functional-group containing compound can be freely selected taking into account the type of functional group to be introduced onto the substrate and the quality of the substrate, and such a compound is widely known in the art. For example, when an amino group is to be introduced onto a glass substrate, a silicon substrate, or other siliconized substrates, a silane compound such as aminoalkyltrialkoxy silane (e.g., aminophrophyltrimethoxy silane or aminophrophyltriethoxy silane) may be used. In addition, in the present invention, for example, when a thiol group is to be introduced onto the substrate, mercaptoalkyltrialkoxy silane (e.g., 3-mercaptophrophyl trimethoxysilane or 3-mercaptophrophyl triethoxysilane) may be used, without being limited thereto.

A method for introducing the functional group by bringing the functional-group containing compound into contact with the substrate is not specifically limited, either, and for example, the functional group may be introduced by dispersing the functional-group compound in a suitable solvent such as water and then dipping the substrate under appropriate conditions.

The second step of the preparation method is a step of introducing the inorganic nanoparticles onto the substrate by bringing the substrate to which the functional group is introduced through the first step into contact with the inorganic nanoparticles.

The type of inorganic nanoparticles has already been described, and the inorganic nanoparticles can be synthesized in various ways known in this field. For example, when metal nanoparticles such as gold nanoparticles are to be used as the inorganic nanoparticles, the nanoparticles may be prepared by reductionism. When magnetic nanoparticles are to be used as the inorganic nanoparticles, the nanoparticles may be prepared by general thermal decomposition.

Examples of a precursor that is applicable to the reductionism may include, but is not limited to, sodium tetrachloroaurate, sodium tetrabromoaurate, tetrachloroauric acid, tetrabromoauric acid, potassium tetrachloroaurate, potassium tetrabromoaurate, tetrachloroauric acid hydrate, or tetrabromoauric acid hydrate. The reductionism may also be carried out by using various reductants known in this field, and for example, the reductant may be ascorbic acid.

A detailed type of nanoparticle precursor that can be used in the thermal decomposition is not specifically limited either, and examples of the precursor may include metal compounds in which metal and —CO, —NO, —C₅H₅, alkoxide, or another well-known ligand are bound. More specifically, various organic metal compounds such as metal carbonyl compounds like iron pentacarbonyl (Fe(CO)₅), ferrocene, or manganese carbonyl (Mn₂(CO)₁₀), or metal acetylacetonate compounds like ferrum acetylacetonate (Fe(acac)₃) may be used. For the nanoparticle precursor, a metal salt including metal ions where metal and well-known anions such as Cl⁻ or NO₃⁻ are bound may be used, and examples of the metal salt may include ferric chloride (FeCl₃), ferrous chloride (FeCl₂), or ferrum nitrate (Fe(NO₃)₃). When an alloy nanoparticle and a complex nanoparticle are to be synthesized, the above-mentioned two or more kinds of metal precursor compounds may be used.

The reductionism or thermal decomposition may be carried out, for example, in various well-known solvents, examples of which may include ether compounds, heterocyclic compounds, aromatic compounds, sulfoxide compounds, amide compounds, alcohol, a hydrocarbon, and/or water. More specifically, for the solvent, an ether compound such as octyl ether, butyl ether, hexyl ether, or decyl ether; a heterocyclic compound such as pyridine or tetrahydrofuran (THF); an aromatic compound such as toluene, xylene, mesitylene, or benzene; a sulfoxide compound such as dimethylsulfoxide (DMSO); an amide compound such as dimethylformamide (DMF); an alcohol such as octyl alcohol or decanol; a hydrocarbon such as pentane, hexane, heptane, octane, decane, dodecane, tetradecane, or hexadecane; or water can be used.

In the preparation method according to the present invention, a method for attaching the inorganic nanoparticles to the substrate is not specifically limited, and for example, the inorganic nanoparticles may be attached by dispersing the inorganic nanoparticles in a suitable solvent such as water and then dipping the substrate under appropriate conditions. Through this process, the inorganic nanoparticles may be effectively attached to the substrate, for example, due to a difference in electric charge density between the functional group, being present on the substrate, and the inorganic nanoparticles.

In the preparation method according to the present invention, after the second step, a step of bringing the substrate to which the inorganic nanoparticles are bound into contact with a target-specific substance may be additionally performed. A detailed type of the target-specific substance has already been described. A method for introducing the target-specific substance to the inorganic nanoparticles of the substrate by bringing the substrate into contact with the target-specific substance is not specifically limited either, and for example, the substrate may be brought into contact with the target-specific substance by using a suitable solvent, such as PBS, as a medium.

The present invention also relates to an analysis apparatus including a bioprobe according to the present invention and a measurement device capable of detecting a signal emitted from the bioprobe.

As mentioned above, in the bioprobe according to the present invention, the inorganic nanoparticles introduced to the substrate serve as a linker to which the target-specific substance such as an antibody can be bound, and they also increase the surface area of the substrate, thus increasing a surface area where a target substance to be detected can contact the substrate, such that the bioprobe can be effectively used in an apparatus for detection, dosing, or analysis of various biomolecules (e.g., a tumor cell, a protein, an antigen, an antibody, and other bio high-polymers) or other chemical substances.

The type of measurement device that can be included in the analysis apparatus according to the present invention is not specifically limited, and a general device known in this field can be used as the measurement device. For example, in the present invention, a device capable of implementing an optical image by detecting a fluorescent signal emitted from the bioprobe can be used. Detailed examples of the analysis apparatus that can be used in the present invention may include, but are not limited to, a Magnetic Resonance Imaging (MRI) device, an epifluorescence microscope, an optical spectrometer, a Charge Coupled Device (CCD), or a CMOS Image Sensor (CIS).

The analysis apparatus according to the present invention may further include a general component such as a housing for receiving the bioprobe and the measurement device.

A method for detecting, dosing, or separating a target substance by using the analysis apparatus according to the present invention is not specifically limited.

Such an analysis method according to the present invention may include, for example, the steps of (1) bringing the bioprobe according to the present invention into contact with an analysis target specimen and (2) detecting a signal emitted from the bioprobe which has passed through step (1).

In the analysis method according to the present invention, a method for bringing the bioprobe into contact with the analysis target specimen is not specifically limited. For example, a specimen, such as a blood plasma, including a target substance to be analyzed (e.g., a tumor cell, a cell, a protein, an antigen, a peptide, DNA, RNA, or a virus) may be brought into contact with the bioprobe under conditions where a target-specific substance on the bioprobe can be bound with the target substance (e.g., conditions where an antigen and an antibody can be bound with each other), and such conditions are well known in the art.

Further, a method for measuring a signal emitted from the bioprobe (especially, the target substance being bound with the target-specific substance of the bioprobe) after brining the specimen including the target substance into contact with the bioprobe is not particularly limited, and for example, general methods using the foregoing various measurement devices may be applied.

In the analysis method according to the present invention, step (1) may further include a step of treating the bioprobe, being in contact with the analysis target specimen, with a fluorescent substance.

This additional step may be performed to further improve the efficiency of detecting the target substance bound with the bioprobe. Examples of the fluorescent substance may include, but are not limited to, one kind or two or more kinds of Hoechst, Fluorescein-5-isothiocyanate (FITC), pyrene, propidium iodide, Rhodamine isothiocyanate (RITC), DAPI, Rhodamine B, Nile red, Texas red, Fluoresceinamine, Alexa flour 488, Alexa Fluor 350, Oregon Green 488, Alexa Fluor 555, Alexa Fluor 594, Alexa Flour 633, Alexa Fluor 647, Alexa Fluor 680, Cy 5.5, Cy 5, and Cy3. In addition, a method for treating the bioprobe (especially, the target substance bound with the bioprobe) with the fluorescent substance is not particularly limited, and a general procedure known in this field can be used.

### MODE FOR INVENTION

Hereinafter, the present invention will be described in more detail with reference to an embodiment thereof, but the scope of the present invention is not limited by the following embodiment.

### Embodiment 1

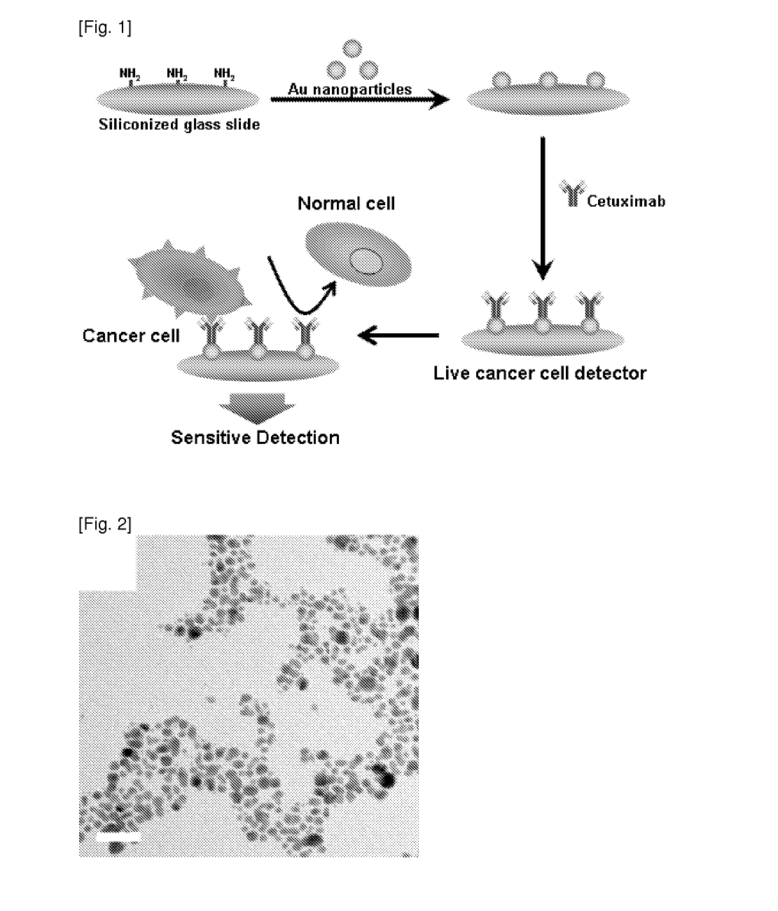

Through a process as shown in FIG. 1, gold nanoparticles are bound to a silicon substrate and an antigen (Cetuximab) is bound to the nanoparticles, thereby preparing a bioprobe. The detailed process is described below.

### Preparation of Gold Nanoparticles

Gold nanoparticles were prepared by reducing 1.0 wt % of tetrachlroloaurate (III) trihydrate (2 ml, Sigma (manufacturer)) for 7 minutes at room temperature in the presence of NaOH (1M, 0.5 ml) and 80 wt % of tetrakis (hydroxymethyl) phosphonium chloride (12 μl, Sigma (manufacturer)) as a reductant. It was verified by use of a Transmission Electron Microscope (TEM) that the prepared gold nanoparticles were mono dispersed particles having an average diameter of about 10 nm (see FIG. 2 where a scale bar is 50 nm).

### Preparation of Bioprobe Including Substrate to Which Gold Nanoparticles are Bound

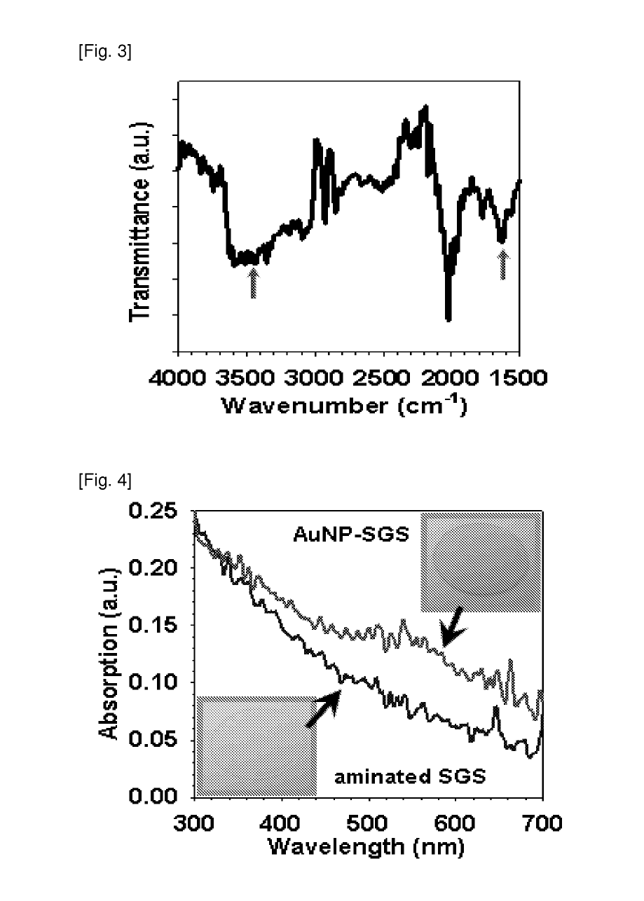

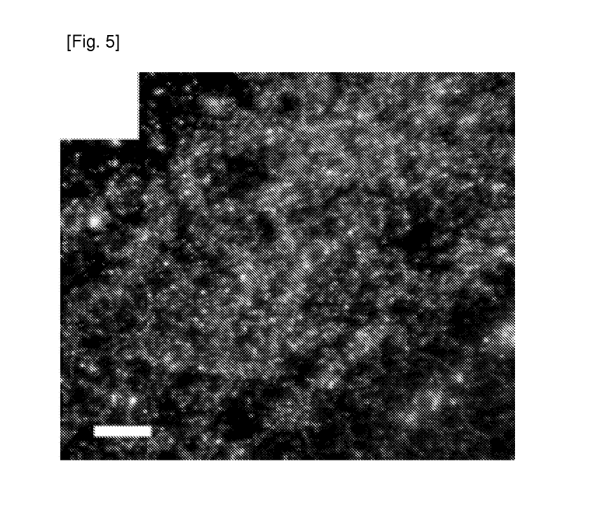

After 3-aminophrophyltrimethoxysilane (100 μl, Sigma (manufacturer)) was dispersed in 5 ml water, siliconized glass slide (Φ=12 mm) was put thereinto and then kept for 24 hours at a temperature of 80° C., thereby preparing a substrate functionalized with an amine functional group. After the prepared substrate was washed with an excessive amount of water and ethanol, it was put into the water where the prepared gold nanoparticles were dispersed (7×1012 particles/ml, 5 ml), and kept for 24 hours under slight stirring. Next, some of the gold nanoparticles which were not bound to the substrate were washed with a sufficient amount of water and ethanol, thereby preparing the substrate on the surface of which the gold nanoparticles are bound. FIG. 3 shows an FT-IR spectrum of the prepared substrate. In FIG. 3, portions indicated by red arrows represent a stretch (3240 cm−1) and a bend (1650 cm−1) of an N—H bond of an amine group, respectively. FIG. 4 shows UV-vis absorption spectrums of an aminated SGS before gold nanoparticles are bound to the amine group and an AuNP-SGS in which the gold nanoparticles are bound to the amine group. As shown in FIG. 4, the absorption spectrum of the AuNP-SGS to which the gold nanoparticles are bound shows a change from the aminated SGS before binding of the gold nanoparticles to the amine group due to a surface plasma effect of the gold nanoparticles deposited on the surface of the substrate, and shows a maximum absorption wavelength of 520 nm. More specifically, the substrate before binding of the gold nanoparticles has a transparent color without exhibiting absorption in the 520 nm wavelength, but the substrate AuNP-SGS to which the gold nanoparticles are bound has a reddish wine color. FIG. 5 shows a light-scattered image of the gold nanoparticles obtained by using a dark field microscope (U-DCW, Olympus (manufacturer)).

### Preparation of Bioprobe in Which Target-Specific Antigen is Bound to Gold Nanoparticles

A target-specific antigen was introduced to the prepared substrate to which the gold nanoparticles were bound. More specifically, the prepared gold nanoparticles-bound substrate was dipped for 4 hours in a 1 ml Phosphate-Buffered Saline (PBS) (10 mM, pH 7.4, Gibco (manufacturer)) where 1 mg Cetuximab (Merck) was dissolved. Next, some of CET which was not bound to the gold nanoparticles was removed with an excessive amount of the PBS solution, thereby preparing the bioprobe to which the target-specific antigen is bound. By using a BAC protein analysis kit (Pierce), the amount of bound CET was dosed.

### Experimental Example 1

### Surface Form Analysis

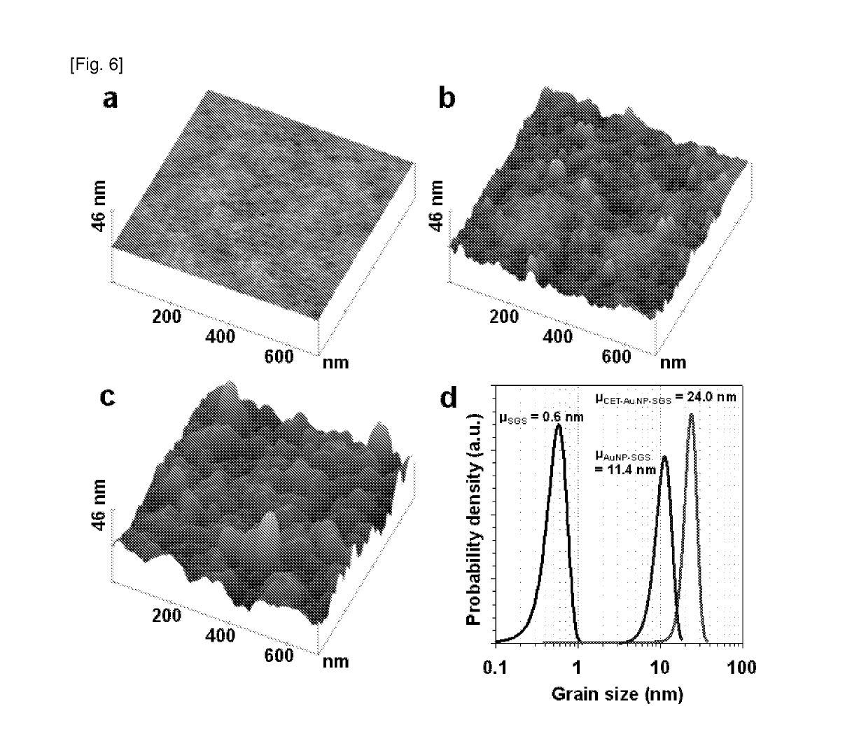

For surface analysis of a substrate at each step of the embodiment, a nanoscope IV controller (Veeco (manufacturer)) was used in tapping mode, in normal air and room temperature conditions. A rectangular AFM silicon cantilever (RTESP TAP300, Metrology Probe, Veeco (manufacturer)) was used for tapping-model AFM, and the same tip and scanning speed were used in analysis of the surfaces of the substrate SGS, the gold nanoparticles-bound substrate AuNP-SGS, and the antigen-bound substrate CET-AuNP-SGS to minimize an error caused by scanning speed or the contact force of the cantilever. AFM data analysis software was used to obtain a histogram of a grain size of the surface, and data collected from AFM was converted by using the program. FIGS. 6A through 6D show the results of the foregoing AFM analysis. FIG. 6A shows a surface state of the substrate SGS to which the gold nanoparticles and the antigen are not bound, FIG. 6B shows a surface state of the gold nanoparticles-bound substrate AuNP-SGS, FIG. 6C shows a surface state of the antigen-bound substrate CET-AuNP-SGS, and FIG. 6D is a grain size diagram of each of the foregoing substrates. As shown in FIGS. 6A through 6C, in the case of the substrate SGS, a height difference of the substrate surface can almost not be observed. However, it can be seen that when the gold nanoparticles and the antigen are bound to the substrate SGS, the surface height and the surface roughness are changed. As can be seen in the grain size diagram (FIG. 6D), a surface size distribution is about 0.6 nm for the substrate SGS, whereas the surface size distribution is about 11.4 nm for the substrate AuNP-SGS and is about 24.0 nm for the antigen-bound substrate (CET-AuNP-SGS).

### Experimental Example 2

### Verification of Detectivity of Bioprobe

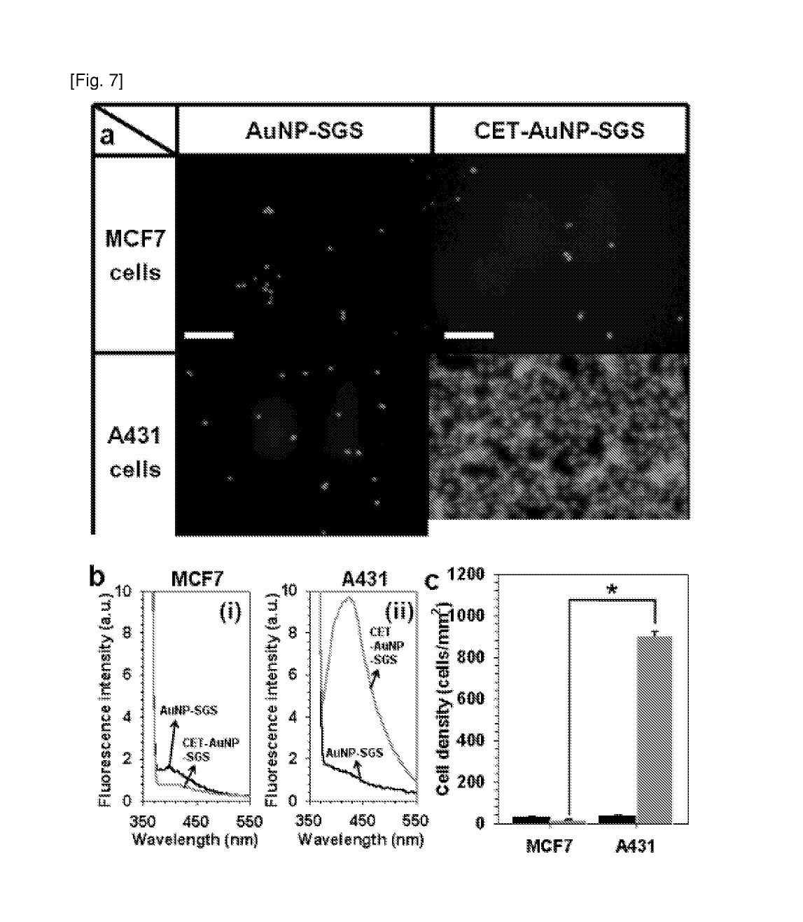

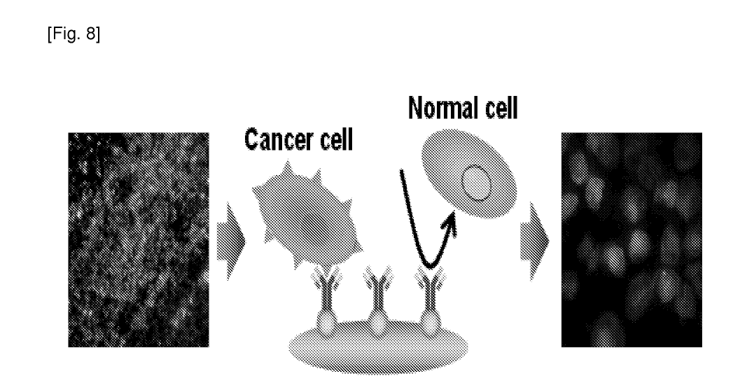

The cancer cell detectivity of the prepared bioprobe was verified by using an epifluorescence microscope (BX-21, Olympus (manufacturer)) and an optical spectrometer (LS-55, Perkin-Elmer). More specifically, after a model cell (MCF7, A431, 1×106 cells/ml) was cultivated, it was treated onto the antigen-bound substrate CET-AuNP-SGS in a 12-well plate (NUNC, 22 mm diameter) for 30 minutes. Next, the resultant was washed 3 times with PBS including 0.2% of Fetal Bovine Serum (FBS) and 0.02% of sodium azide, and then cultivated in a darkroom for 10 minutes at a temperature of 4° C. with Hoechst 33258 (λexcitation=350 nm, λemission=461 nm). Thereafter, the cultivation well was washed 3 times with an excessive amount of PBS and the detectivity of an epithelial cancer cell was verified by using the epifluorescence microscope. The detectivity of a liver cancer cell was measured by using the spectrometer. FIGS. 7A through 7C and 8 show the foregoing analysis results. FIG. 7A shows epifluorescence microscope images of the substrate CET-AuNP-SGS and the substrate AuNP-SGS treated with MCF7 and A431 cells, respectively. It can be seen from FIG. 7A that the substrate CET-AuNP-SGS can specifically detect the epithelial cancer cell A431 (in FIG. 7A, the blue spot indicates a cell nucleus which is fluorescent stained by Hoechst 33258 and the scale bar is 100 μm). In FIG. 7B, (i) shows a fluorescent spectrum of light emitted from the substrate CET-AuNP-SGS and the substrate AuNP-SGS treated with the MCF7 cell, (ii) shows the A431 cell after excitation in a wavelength of 350 nm, and (iii) shows cell densities of the substrate CET-AuNP-SGS (red) and the substrate AuNP-SGS (black) treated with the MCF7 and A431 cells, respectively. As can be seen in FIG. 7B, the substrate CET-AuNP-SGS treated with the A431 cell exhibits a cell density that is about 54 times higher than that of the substrate CET-AuNP-SGS treated with the MCF7 cell.

What is claimed is:

1. An analysis method comprising the following steps of:
(1) bringing a bioprobe into contact with an analysis target specimen, with the proviso that the bioprobe comprises (a) a substrate; (b) inorganic particles of an average diameter of 1 nm-20 nm attached to the surface of the substrate, the number of attached inorganic particles is 10-50 per one square micron of the substrate; and (c) a target-specific substance that is specifically bindable to a tumor marker and that is bound to the inorganic particles of the substrate, the average roughness of the substrate to which the inorganic particles are attached is 10 nm-1 um; and
(2) detecting a signal emitted from the bioprobe which has passed through step (1).

2. The analysis method of claim 1, wherein the analysis target specimen comprises a tumor cell.

3. The analysis method of claim 1, wherein step (1) further comprises a step of treating the bioprobe, being in contact with the analysis target specimen, with a fluorescent.

4. The analysis method of claim 1, wherein the substrate is glass, a silicon substrate, quartz, metal, or a high-polymer film.

5. The analysis method of claim 1, wherein the inorganic nanoparticles are metal nanoparticles or magnetic nanoparticles.

6. The analysis method of claim 5, wherein the metal nanoparticles are one or more selected from a group consisting of gold nanoparticles, platinum nanoparticles, silver nanoparticles and copper nanoparticles.

7. The analysis method of claim 5, the magnetic nanoparticles are metal substances, magnetic substances, or magnetic alloys.

8. The analysis method of claim 1, wherein the nanoparticles are attached to the surface of the substrate by using one or more functional groups selected from a group consisting of an amine group and a thiol group as a medium.
